(19) United States
(12) Patent Application Publication
(10) Pub. No.: US 2021/0212593 A1
(43) Pub. Date: Jul. 15, 2021

(54) Disposable Differential-Pressure-Type Respiratory Flow Device

(71) Applicant: GoldverTech Systems Co. Ltd., Chupei, Hsin Chu County (TW)

(72) Inventor: Mengbiao Xu, Wuxi (CN)

(21) Appl. No.: 16/076,013

(22) PCT Filed: May 17, 2016
(86) PCT No.: PCT/CN2016/082333

§ 371 Date: Aug. 7, 2018

(51) Int. Cl. A61B 5/087

(52) U.S. Cl. CPC A61B 5/087; A61B 5/091

(57) ABSTRACT

Disclosed is a disposable differential-pressure-type respiratory flow device, which is provided with a fixed pipe (10). The front and rear sections of the fixed pipe (10) are divided into a drainage section (11) and an engaging section (12). Two differential pressure measuring holes (121) are provided at front and rear portions of the engaging section (12). A pluggable mouthpiece (20) is attached to the engaging section (12) of the fixed pipe (10). A throttling element (24) is provided in the mouthpiece (20). Corresponding to the position of the two differential pressure measuring holes (121), two rows of pressure guide holes (211) arranged annularly and corresponding to the two pressure difference measuring holes (121) are provided in the front and rear of the throttling element (24). The throttling element (24) is interposed between the two rows of pressure guide holes (211). The above-mentioned respiratory flow device forms the throttling element (24) in the disposable mouthpiece (20), such that the specifications of the throttling element (24) can vary with different mouthpieces (20), anil thus the measurement range is greater, and suitable for accurate measurement of respiratory flow data of users of various body types.

### FIELD OF THE INVENTION

The present invention relates to a disposable differential-pressure-type respiratory flow device, and more particularly to disposable differential-pressure-type respiratory flow device that has throttle pieces and single-use mouth with the components of the mouth can be combined with the mouth with components to replace the differential pressure breathing flow device.

### DESCRIPTION OF RELATED ART

As a result of industrialization, factories, vehicles, exhaust emissions on the environment serious air pollution asked question, triggering many human respiratory diseases, such as asthma, chronic bronchitis and other diseases.

For medical personnel to determine whether the patient has respiratory problems, the respiratory function measuring instruments will be used to measure the lung function, such as differential pressure flow meter, is to provide breathing channel for patients to place the orifice plate as a section Flow, the use of differential pressure sensor to measure the air flow through the orifice plate before and after the pressure difference to the pressure difference data with the orifice area, fluid density and other known data to calculate the lungs respiratory flow rate, flow and lung Of the volume, medical staff to the value of these lung functions and the standard value of comparison, you can see the health of lung function or not.

The differential pressure flowmeter can be used to measure lung function, but its use has technical limitations. Due to the fixed and permanent size of the throttling element provided in the pressure channel of the existing differential pressure flowmeter, the flow rate of the same person when breathing normally and breathing strongly differs, Breathing individual differences greater, adults and children, brawny and sick women's respiratory flow difference is not only one or two times the difference only, so the fixed size of the flow control allows the existing differential pressure flow meter in use There are narrow limits of measurable flow range. Therefore, with the same fixed-flow ratio differential pressure flow meter, it is difficult to complete a variety of different physical conditions of human respiratory measurement, prone to error in the measurement.

Gauge, it is difficult to complete a variety of different physical conditions of human respiratory measurement, prone to error in the measurement. In addition, the human exhaled gas temperature and humidity than the environment, when the gas is blown into the existing differential pressure flow meter pipe, once hit the cooler pipe wall, the higher humidity gas will condense into water droplets. In addition, some people are likely to flow out of breath when exhaled, both of the above conditions will obstruct the existing differential pressure flow meter pressure channel, so that the results of the measurement error, if you want to clean up the pipeline of the pressure channel Increased trouble.

### Content of the Present Invention

The existing differential pressure flow meter pipeline has a narrow flow range, as well as cannot rule out the saliva, condensing water droplets and make the measurement error results. To this end, the main object of the present invention is to combine the throttling member with the disposable mouthpiece assembly so as to be able to adapt to measuring respiration of different people and ensure a wide measurement range and accuracy. And further to identify the structure and fabric structure, to automatically identify the throttle size, to prevent moisture condensation, droplets clogging the channel, affect the measurement accuracy and eliminate the secondary purpose of pipeline obstruction.

To overcome the shortcomings, the present invention provides disposable differential-pressure-type respiratory flow device to mitigate or obviate the aforementioned problems, and has:

a fixed pipe, being a straight pipe body with front and rear ends, the front and rear ends of the pipe are open the front section of the fixed pipe is a drainage section and the rear section is an engaging section which penetrates the two pressure difference measuring holes in the radial direction at the front and rear positions around the engaging section; a fan unit, the fan unit mounted in the body near the air inlet, the fan unit being rotated relative to the body to enable external air to flow into the body via the air inlet and flow out of the body via the air outlet, and having an external surface being obliquely divergent from the air inlet to the air outlet to form an oblique passage between the internal surface of the body and the external surface of the fan unit; and

a mouth tube, a cannula section and a mouth bite mouth section are arranged on the front side and the back side, respectively, the cannula section of the mouth-position tube is inserted in the engaging section of the fixing tube in a pluggable form, The position of the poor measuring hole, the front and rear positions around the cannula section are respectively provided with more than one pressure guide holes in a round way, the pressure guide holes on the front side communicate with the pressure measuring holes on the front side, the rear side Of the pilot pressure hole and the back of the differential pressure measurement hole communicating in the cannula section is provided with a throttle member, the throttle member blocking the front and rear sides of the pressure port between the different specifications Ports have different degree of throttle throttling the size of the pieces.

Further, the two differential pressure measuring holes of the present invention are formed on the side around the engaging section, the inner diameter of the drainage section is smaller than the inner diameter of the engaging section, and the inner peripheral surface of the drainage section and the engaging section A shoulder portion is formed between the inner peripheral surfaces; the front end of the cannula segment abuts against the shoulder portion, and the inner diameter of the cannula segment is equal to the inner diameter of the drainage segment. Still further, in the present invention, one or more working holes are arranged in a straight line arranged at the front and rear intervals on the side around the engaging section, and the positions of the working holes are matched with each other selectively or not on the outer peripheral surface of the inserting section In some ways, a concavo-convex structure is formed. There are two or more combinations of the number and the positions of the concavo-convex structure, and each combination corresponds to the specification of one type of throttle.

Preferably, according to the present invention, an insertion groove extending in the front-back direction is recessed formed on the inner peripheral surface of the fixing pipe corresponding to the position of the inner end of each of the working holes, the insertion groove is formed with an opening at the rear end face of the fixing pipe, The working holes are communicated with each other; each of the concave-convex structures is a convex structure, and each of the convex structures can slide along the inserting groove.

Preferably, the position corresponding to the two pressure-difference measuring holes according to the present invention is respectively formed with an annular groove in an annular shape on the inner circumferential surface of the fixed pipe, each of the annular grooves and the corresponding differential pressure measuring hole.

Preferably, the present invention penetrates a pressure measuring hole on one side around the drainage section, and the inner end of the pressure measuring hole communicates with the interior of the drainage section.

Preferably, in the invention, a fabric is adhered on the surface of the cannula section, and the fabric covers the pressure guide holes the driving unit has a protecting cover securely connected to the driving motor and the inlet shield to mount around the driving wheel and the transmitting belt.

Preferably, the invention corresponds to the positions of a plurality of pressure guide holes which are arranged around the front and the back sides, two shallow grooves are concavely arranged on the outer circumferential surface of the cannula section and a fabric is pasted on each shallow groove, Coverage of the pressure port.

Preferably, in the invention, a filter layer is embedded inside the mouthpiece segment.

Preferably, in the present invention, a straight guiding structure is formed between the inner peripheral surface of the engaging section and the outer peripheral surface of the cannula section. The guiding structure includes a guiding groove and a guiding protrusion which can slide along the guiding groove.

When the invention is used, the fixed pipe is installed on the differential pressure flowmeter as a part of the fixed pipeline, and the differential pressure sensor of the differential pressure flowmeter is connected to the two differential pressure measurement holes of the fixed pipe. When the user wants to measure the respiration, since the differential pressure flowmeter is used to measure the respiration of different people, for the sake of hygiene, each user needs to use an unused mouth Tube, insert the mouth tube section of the mouth tube into the engaging section of the fixed pipe, and make the mouth tube located at the front and back sides of the pressure-guiding hole and the corresponding pressure-measuring hole communicating with the user Fixed pipe blowing, inhalation for measurement.

When the air exhaled by the user passes through the throttling member in the cannula section, a pressure difference will be generated due to the airflow passing through the throttling member. At this time, the differential pressure sensor can measure through two pressure-difference measuring holes The value of the pressure difference, with the throttle parts specifications (for example, in the fixed pipe surface indicates the specifications of the internal throttle, allowing users to manually read the known), gas density, time and other known relevant parameters, let Differential pressure flow meter to calculate the respiratory flow, flow and volume and other lung function values. After the user has measured the respiration, the mouthpiece is withdrawn and discarded for non-repeated use.

The main effect of the present invention is that since the throttle member is formed in the disposable mouthpiece, there is no need to change the fixed pipeline, so that different orifice sizes can be throttled in different orifice tubes Piece, to expand the scope of the throttling pieces of specifications, increase the differential pressure flow meter can measure the breathing range, with different populations appropriate respiratory flow to select the appropriate mouthpiece, the respiratory flow measurement obtained more accurate measurement results.

The invention further has a working hole on the fixed pipe and a corresponding concave-convex structure matched with the working hole on the mouth-piece pipe to provide the effect of automatically recognizing the size of the orifice plate in cooperation with the detection of the micro-switch. Further, a filter layer is arranged in the mouthpiece segment to filter the filter water, and the covered fabric is provided in each of the pressure guide holes. Since the fabric does not have thermal conductivity, wet air is not easily formed on its surface to form dew so as to avoid saliva or Condensation block the cannula section or the pressure guide hole, so that the pressure measurement hole output airflow pressure from saliva or condensation of water droplets.

According to the above-mentioned technical features, the disposable differential-pressure-type respiratory flow device for a grain dryer in accordance with the present invention has the following advantages and effects:

First Increase useful life: the disposable differential-pressure-type respiratory flow device of the invention, divergent spatial morphology of the oblique passage between the guiding sheath of the body and the fan of the fan unit can enable the air to flow in the oblique passage without vibrating or damaging the fan unit during the rotation process. In addition, the driving unit is mounted on the body instead of mounting in the body to avoid affecting by the ashes or impurities and to reduce the temperature of the driving motor, and the life of the fan unit and the driving motor can be prolonged.

Second Cost savings: the disposable differential-pressure-type respiratory flow device of the invention in use, the damage probability and number of replacement of the fan of the fan unit may be reduced. Furthermore, since the driving motor s mounted on the body rather than in the body and the driving motor can be maintained or repaired conveniently, and this can reduce the time and cost of use.

Third Multiple collecting times and cleaning effect: the disposable differential-pressure-type respiratory flow device for a grain dryer of the invention, the guiding sheath of the body has a divergent internal surface, and the fan of the fan unit has a an external surface being obliquely divergent from the inlet shield to the exhaust housing, and this enable an annular oblique passage to form between the guiding sheath of the body and the fan of the fan unit. When the air flows in the body and flows to the oblique passage, the guiding vanes of the rectifier seat enable the air to flow along an internal surface of the body in a centrifugal rotating type, and this enable ashes or impurities in the air to accumulate on the internal surface of the body to reduce the ashes or impurities in the air to provide an ash-collecting and air-cleaning effect. Furthermore, the recovery unit can collect the ashes or impurities in the air one more time, and this can improve the cleaning effect to the air.

### BRIEF DESCRIPTION OF THE DRAWINGS

FIG. 1 is an exploded top side view of a first embodiment in accordance with the present invention.

FIG. 2 is a side view in partial section of the first embodiment of the present invention.

FIG. 3 is a cross sectional and exploded side view of the first embodiment of the present invention.

FIG. 4 is a cross sectional side view of a mouth tube of a second embodiment of the present invention.

FIG. 5 is a cross sectional side view of a mouth tube of a third embodiment of the present invention.

FIG. 6 is a cross sectional top side view of a mouth tube of a fourth embodiment of the present invention.

[Description of the Reference Numbers]

| | | | |
|---|---|---|---|
| 10 | fixed pipe | 11 | drainage section |
| 111 | pressure measurement hole | 12 | engaging section |
| 121 | differential pressure | 122 | annular groove |
| | measuring hole | | |
| 13 | ring-shaped shoulder | 14 | guide groove |
| 15 | working hole | 16 | insertion groove |
| 20 | mouthpiece | 21 | cannula section |
| 211 | pressure guide hole | 22 | bevel section |
| 23 | mouthpiece section | 24 | throttling element |
| 241 | circular hole | 25 | fabric |
| 26 | guiding protrusion | 27 | concavo-convex structure |
| 28 | filter layer | 29 | shallow groove |

### DETAILED DESCRIPTION OF PREFERRED EMBODIMENT

To be able to learn more about the technical features and practical efficacy of the present invention, in accordance with the instructions, to implement further the figures shown in the preferred embodiment, details are as follows:

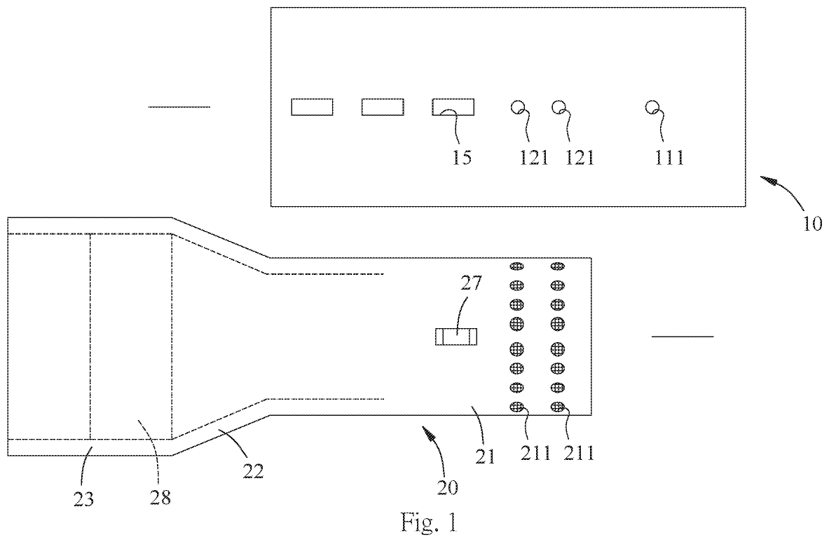

The present invention provides a disposable differential pressure breathing flow device, please refer to FIG. 1 to FIG. 3 first, the structure of the preferred embodiment includes a fixed pipe 10 and a mouthpiece 20 that is coupled to the fixed pipe 10 in a pluggable manner.

The fixed pipe 10 is a plastic circular straight pipe body, which is a plastic circular straight pipe body, which is transversely extending in the front-back direction and has both front and rear ends open as in the present preferred embodiment End of the fixed pipe 10 is divided into a drainage section 11 and an engaging section 12, the drainage section 11 and the engaging section 12 the same outer diameter, the drainage section 11 is smaller than the inner diameter of the engaging section 12 The inner diameter of the fixed pipe 10 is located between the drainage section 11 and the engaging section 12 forms a ring-shaped shoulder 13.

Around the drainage section 11, as in the preferred embodiment, a pressure measurement hole 111 is radially penetrated through the top of the drainage section 11, and the inner end of the pressure measurement hole 111 is communicated with the interior of the drainage section 11. The front and rear positions around the front side of the engaging section 12, as in the present preferred embodiment, are respectively penetrated through a differential pressure measuring hole 121 in the radial direction at the top and front and rear positions around the front side of the engaging section 12, The inner ends of the differential pressure measuring holes 121 communicate with the interior of the engaging section 12 respectively. Corresponding to the inner ends of the two differential pressure measuring holes 121, an annular groove 122 is recessed on the inner circumferential surface of the fixed pipe 10 in an annular shape. The inner end of each of the differential pressure measuring holes 121 is flush with the top of each annular groove 122 side of the same.

In the inner peripheral surface of the engaging section 12, as in the present preferred embodiment, a guide groove 14 is recessed in the front and rear direction on the inner peripheral surface of the bottom side of the engaging section 12. The front end of the guide groove 14 is located in the two annular grooves 122 and the rear end of the guide groove 14 forms an opening in the rear end surface of the fixing pipe 10.

Around the bridging section 12, as in the present preferred embodiment, is the top side around the bridging section 12, and three working holes 15 are longitudinally penetrated by the arrangement of the front and rear compartments. The three working holes 15 are arranged in the front—The inner peripheral surface of the fixed pipe 10 is provided with an insertion groove 16 extending in the front-rear direction. The front end of the insertion groove 16 is located behind the two annular grooves 122, and the rear end of the insertion groove 16 forms an opening in the rear end surface of the fixing pipe 10. The insertion groove 16 communicates with the inner ends of the three working holes 15 respectively.

The mouth-piece tube 20 is a circular plastic straight tube body with a narrow front width and a narrow width. As shown in the preferred embodiment, the mouth tube 20 is a plastic structure with open ends at the front and rear ends. The front, middle and rear positions of the mouthpiece 20 are divided into a cannula section 21, a bevel section 22 and a mouthpiece section 23. For the purpose of hygiene, the mouth-mounted tube 20 is usually only for one-time use by a user, among them:

The cannula section 21 is a straight body, and the mouthpiece cannula 20 is inserted into the cannula section 21 from the rear into the engaging section 12 of the cannula section 10. The front end of the cannula section 21 abuts against the shoulder of the cannula 10 Ministry 13. The outer diameter of the cannula section 21 is equal to the inner diameter of the engaging section 12 and the inner diameter of the cannula section 21 is equal to the inner diameter of the drainage section 11 so that the cannula section 21 is inserted into the fixing tube 10 after the bridging section 12, a passage with uniform inner and outer diameters is formed inside the fixed pipe 10.

In accordance with the position of the two differential pressure measuring holes 121, one or more pressure guide holes 211 are drilled in the circumferential front and rear positions on the front side of the cannula section 21, as in the present preferred embodiment A plurality of pressure guide holes 211 are provided around the front and rear positions of the front side of the cannula section 21 respectively and are arranged at intervals, the plurality of pressure guide holes 211 communicates with the annular groove 122 on the front side, and the plurality of pressure guide holes 211 on the rear side communicate with the annular groove 122 on the rear side.

The plate 24 serves as a throttling member, and the orifice plate 24 divides the plurality of pressure-guiding holes 211 on the front side into

A pressure guide hole 211, a circular hole 241 is formed in the middle of the throttling element 24, as in the present preferred embodiment

The center of the aperture 241 is concentric with the axis of the cannula section 21. With the front side of the plurality of pressure guide hole 211 and the rear

The positions of the plurality of pressure guide holes 211 on the side are respectively on the circumferential surface of the cannula section 21, as in the present preferred embodiment. A fabric 25 is adhered to the inner peripheral surface of the cannula section 21, and the fabric 25 covers a plurality of pressure guide holes 211. The fabric 25 does not have thermal conductivity, and moisture is less likely to form dew on its surface.

A guiding protrusion 26 protrudes from the outer circumferential surface of the cannula section 21 in cooperation with the guide groove 14 of the fixing tube 10. When the cannula section 21 is inserted into the fixing tube 10, the guiding projection 26 can extend along the guide groove 14 is slid forward, and when the cannula section 21 is inserted into the engaging section 12, the guide projection 26 is positioned at the front end of the guide groove 14, and the guide projection 26 ensures that the cannula section 21 is inserted into the fixing tube 10 in the process can maintain a straight-forward state, to avoid the fixed pipe 10 is rotated.

A concavo-convex structure 27 is selectively formed or not provided on the top of the outer peripheral surface of the cannula section 21 in accordance with the position of each of the working holes 15 of the fixing tube 10. Each of the concavo-convex structures 27 may be a convex structure or the recessed configuration, as in the present preferred embodiment, is selected only below the working aperture 15 on the foremost side. A concavo-convex structure 27 which is a protruding structure slidable forward and backward along the insertion groove 16 and cooperating with the micros-witch additionally mounted on each of the working holes 15 to detect the presence or absence of the Convex structure 27.

The configuration from the state of the three working holes 15 are not provided under the concavo-convex structure 27 to the three working holes 15 are provided under the concavo-convex structure 27 state, the present invention can be provided at the top of the engaging section 122 has eight different concavo-convex structure 27 number In combination with the position, the same combination of the number and position of the concavo-convex structure 27 corresponds to the same type.

The size of the orifice plate 24, that is, the combination of the number and positions of the eight kinds of concavo-convex structures 27 makes it possible to distinguish the diameters of the eight kinds of orifices 241. A list of the specifications for the combination of the number and positions of the various relief structures 27 is as follows, where 0 means not provided and 1 means that the specifications of the orifice plate 24 provided with, for example, the mouthpiece 20 of this preferred embodiment fall within the following specifications 2:

The inclined section 22 is an annular shape with a narrow front width and a rearward width. A front end edge of the inclined section 22 is connected to a rear end edge of the cannula section 21. The mouth section 23 is a straight body with a diameter larger than that of the cannula section 21. The front edge of the mouthpiece section 23 is connected to the rear end edge of the inclined section 22. The mouthpiece section 23 serves to allow a user's mouth to grip the mouthpiece 20. The plug is provided with a filter layer 28, which may be constructed of a fiber filler layer such as wood fiber or plastic fiber. The filter layer 28 allows the air to pass through and blocks the saliva from entering the cannula section 21.

| | The | The | The |
|---|---|---|---|
| | concavo-convex | concavo-convex | concavo-convex |
| | structure 27 on | structure 27 on | structure 27 on |
| Specifications | the front side | the middle | the rear side |
| 1 | 0 | 0 | 0 |
| 2 | 1 | 0 | 0 |
| 3 | 0 | 1 | 0 |
| 4 | 0 | 0 | 1 |
| 5 | 1 | 1 | 0 |
| 6 | 1 | 0 | 1 |
| 7 | 0 | 1 | 1 |
| 8 | 1 | 1 | 1 |

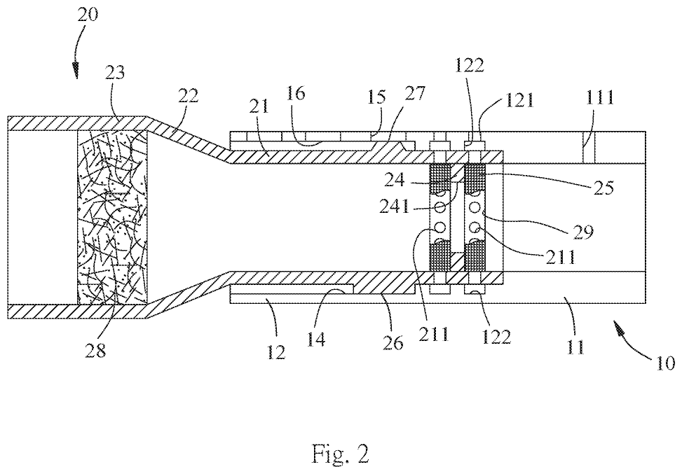

When the present invention is installed as a part of a pipeline in a differential pressure type flow meter, as shown in FIG. 2, the first preferred embodiment is based on the connection of two differential pressure measuring holes 121 separated by the front and rear of the orifice plate 24 Pressure sensor, and the pressure measurement hole 111 is located in the drainage section 11 is connected to the pressure sensor, and in the corresponding fixed pipe 10 of the three working holes 15 is provided with three micro-switches, each micro-switch is provided with a transmission assembly Such as a pin, a button, a lever, a roller and other transmission components. When the mouth-piece tube 20 is inserted into the fixing tube 10, the transmission component can detect the presence or absence of the concave-convex structure 27 under the working hole 15, The size of the throttling element 24 of the mouthpiece 20 to be inserted is determined so as to obtain the data of the diameter of the aperture 241 of the mouthpiece 20.

The pressure sensor of the differential pressure flow meter is used to measure the absolute pressure of the airflow passing through the fixed pipe 10 to calculate the gas density. The differential pressure sensor is used to measure the flow of the energy before and after passing through the orifice plate 24 The difference in pressure is measured by the sensor using data matching the orifice of the orifice plate 24 diameter, gas density, time and other known relevant parameters can be calculated by the mouth of the mouth of the tube 20 is inserted into the cannula section 21 and the fixed pipe 10 air flow, flow rate and volume.

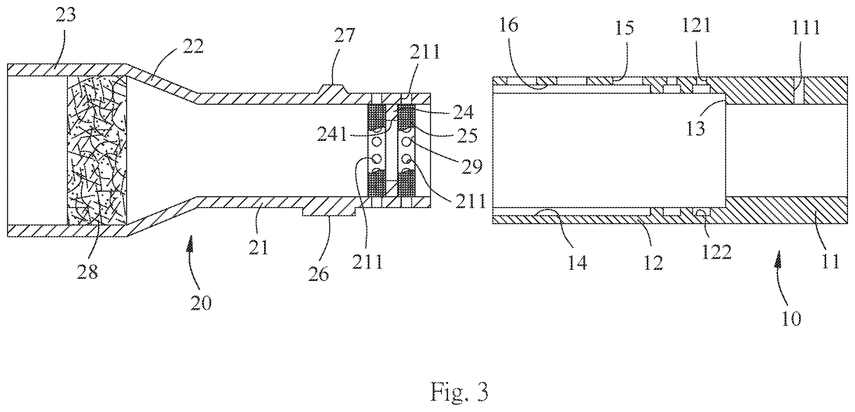

When the user applies the present invention, as shown in FIG. 2 and FIG. 3, the throttling element 24 having a larger aperture 241 according to the specifications of the throttling element 24 suitable for the user is suitable for measuring adult or strong body, orifice plate 24 with smaller orifices 241 is suitable for measuring young or infirm populations, selecting a mouthpiece 20, as in the present preferred embodiment, is a mouthpiece 20 of a selected size 2, and then the cannula section 21 of the mouthpiece 20 is inserted into the engagement section 12 of the fixation tube 10, The guide protrusion 26 on the bottom side of the pipe section 21 enters along the guide groove 14 of the engaging section 12 to exert the guiding effect to prevent the cannula section 21 from rotating during the insertion of the engaging section 12.

After the cannula section 21 of the mouthpiece 20 is inserted into the fixing tube 10, as shown in FIG. 2, the concave-convex structure 27 is located below the working hole 15 on the front side. At this time, the three microswitches can detect the front There is a concavo-convex structure 27 below the working hole 15 and there is no concavity and convexity below the middle and rear working holes 15 in the configuration 27, it is possible to determine in an automated manner that the orifice plate 24 of the mouthpiece 20 is the orifice plate 24 of size 2 and the diameter of the orifice 241 of the mouthpiece 20 is read out.

When the mouthpiece 20 is inserted into the fixing tube 10, a plurality of pressure-guiding holes 211 on the front side and a plurality of pressure-receiving holes 211 on the rear side are respectively passed through the annular grooves 122 on the front and rear sides to be connected to the front and rear sides Of the differential pressure measuring hole 121. So that the user bites the mouthpiece section 23 and blows into the mouthpiece 20. When the humidified air exhaled by the user passes through the throttling element 24 in the cannula section 21, the above-mentioned differential pressure sensor can transmit two differential pressure measuring holes 121 to measure the pressure difference before and after the air exhaled by the user passes through the orifice plate 24.

Since the mouthpiece 20 of the present invention is of a disposable configuration and the throttling element 24 is positioned at the disposable mouthpiece 20, Different sizes of orifice plate 24, with a variety of restrictions on the specifications to adapt to different body size or physical ability. The user's breath, can increase the differential pressure flowmeter to measure the breathing range, and to obtain more accurate measurement results.

In the invention, the filter layer 28 is arranged in the mouthpiece segment 23 to filter the saliva breathed by the user into the cannula segment 21 to prevent the saliva from entering the cannula segment 21 and block the pressure guide holes 211; The fabric 25 of the hole 211 can prevent the damp air from condensing on the cooler wall of the pressure guide hole 211 and avoid the dewdrops block the pressure guide hole 211.

Further, when each pressure guide hole 211 is engaged with the pressure differential measuring hole 121 of the fixed pipe 10, the plurality of pressure guide holes 211 on the front and back sides are connected to the pressure measuring holes 121 through the corresponding annular grooves 122. In this way, when several of the pressure guide holes 211 are blocked, other annular grooves 122 remain pass, will not affect the pressure differential measuring hole 121 output airflow pressure results.

In the present invention, the design of the three pressure relief holes 211 for avoiding saliva or damp air prevents the obstruction of the pipe of the mouth-piece pipe 20 and the fixed pipe 10 to a great extent and reduces the error of the differential pressure sensor measurement, effectively improve the accuracy of differential pressure flowmeter measurement.

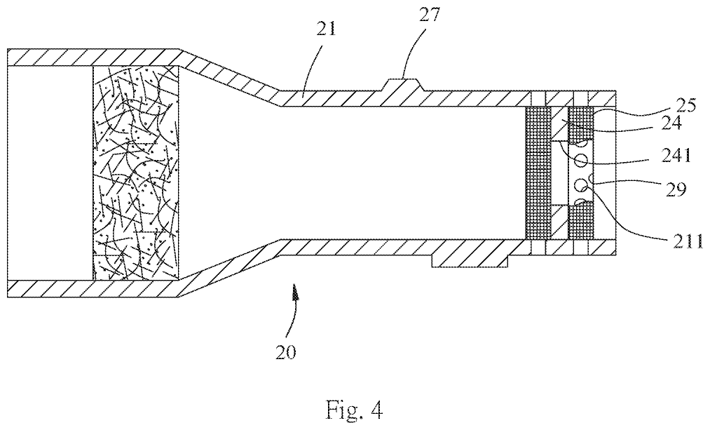

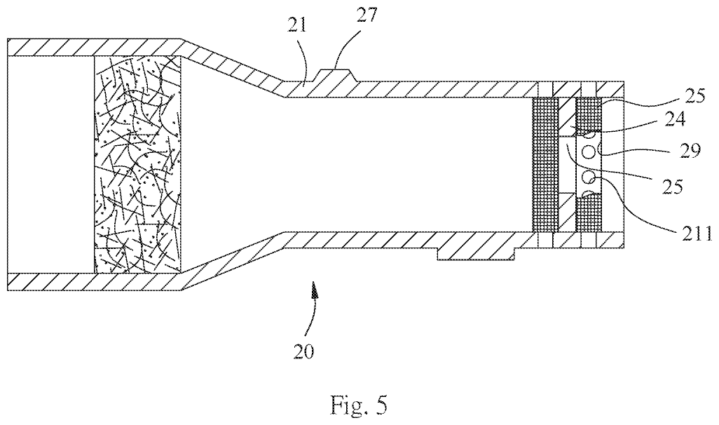

The mouthpiece 20 of the present invention can have a variety of orifice plate 24 specifications. As shown in FIG. 4, the second preferred embodiment of the present invention is provided with a concavo-convex structure 27 only at the position below the intermediate working hole 15 of the cannula section 21, the mouth-piece tube 20 belonging to the standard 3, the second The aperture 241 of the throttling element 24 in the preferred embodiment is smaller than the aperture 241 of the first preferred embodiment. As shown in FIG. 5, the third preferred embodiment of the present invention is provided with a concavo-convex structure 27, a mouth-piece tube 20 belonging to a size 3, only the lower position of the cannula section 21 corresponding to the rear side working hole 15, In the preferred embodiment, the aperture 241 of the throttling element 24 is smaller than the aperture 241 of the second preferred embodiment. The size ratios of the throttling elements 24 of the other specifications of the aperture 20 are similar.

In addition to the above first preferred embodiment of the present invention, the front and rear two guide grooves 211 are arranged around the guide hole 211, and two annular grooves 122 communicating with the two surrounding guide grooves 211 are provided in the fixing tube 10 The two annular grooves 122 may not be provided. In this case, as long as one of the circumferentially arranged row of pressure guide holes 211 communicates with the pressure differential measuring hole 121, the outer ends of the remaining differential pressure measuring holes 121 are the inner periphery of the engaging section 12 is closed.

In the present invention, in addition to the first preferred embodiment described above, a plurality of pressure guide holes 211 are provided in the front and rear positions around the front of the cannula section 21, respectively, and the fixing pipe 10 may be provided without Two annular grooves 122, and corresponding to the two differential pressure measuring holes 121 of the fixed pipe 10, only the cannula section 21, the top front and rear positions are respectively penetrated by a pressure guide hole 211, and each pressure guide hole 211 is directly connected with each pressure measuring hole 121.

In addition, when the concavo-convex structure 27 provided in the mouthpiece 20 has a convex structure, the function of preventing the mouthpiece 20 from rotating due to the protruding structure being capable of sliding back and forth in the insertion groove 16, the mouth tube 20 may not be provided with the guide protrusion 26. In addition, the guide protrusion 26 cooperates with the guide groove 14 to form a rectilinear guide structure between the mouth-piece tube 20 and the fixing tube 10. The position of the guide protrusion 26 and the guide groove 14 can be mutually Change, you can still achieve a linear guide effect.

When there are fewer or more specifications of the orifice plate 24 of the mouthpiece 20, the number of the working holes 15 on the fixing tube 10 can be reduced or increased and correspond to the positions of the respective working holes 15. After inserting the fixation The insert section 21 of the tube 10 is formed with or without the above-described concavo-convex structure 27, and the presence or absence of the concavo-convex structure 27 is detected by each microswitch through each of the working holes 15 An orifice tube 20 having orifice plates 24 of various sizes.

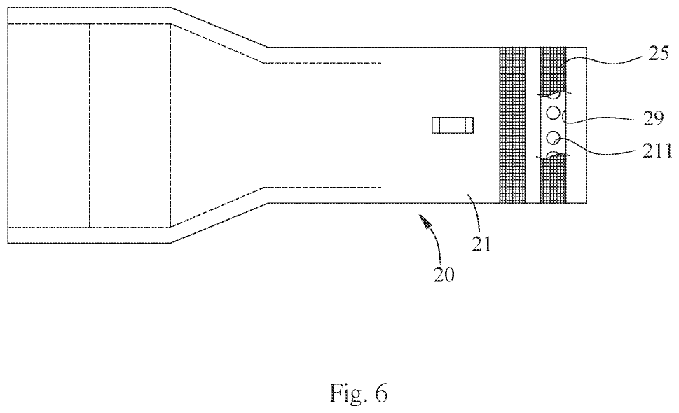

In addition to the above preferred embodiment of the present invention, a fabric 25 is adhered to the inner peripheral surface of the cannula section 21, and the fabric 25 covers a plurality of pressure guide holes 211. As shown in FIG. 6, the fourth preferred embodiment, But also corresponding to the position of the two surrounding pressure guide holes 211, two shallow grooves 29 are formed around the outer periphery of the cannula section 21 of the mouthpiece 20, and the fabric 25 is attached to each of the shallow grooves 29 So as to cover each of the pressure guide holes 211 with the fabric 25, so that each fabric 25 does not protrude from the outer peripheral surface of the cannula section 21.

Even though numerous characteristics and advantages of the present invention have been set forth in the foregoing description, together with details of the structure and features of the invention, the disclosure is illustrative only. Changes may be made in the details, especially in matters of shape, size, and arrangement of parts within the principles of the invention to the full extent indicated by the broad general meaning of the terms in which the appended claims are expressed.

1. A disposable differential-pressure-type respiratory flow device, characterized in that the disposable differential-pressure-type respiratory flow device having:
a fixed pipe, being a straight pipe body with front and rear ends, the front and back ends of the fixed pipe are open ends, the front section of the fixed pipe is a drainage section and the rear section is an engaging section, The convergence section around the front and rear position along the radial through the two differential pressure measurement hole; and
a mouth tube, a cannula section and a bite mouth section are respectively arranged on the front side and the back side, the cannula section of the mouth-piece tube is inserted in the engaging section of the fixed pipe in a pluggable faun, Two differential pressure measurement hole position, before and after the cannula segment around the front and rear positions respectively arranged in a manner to pierce more than one pressure guide hole, the front side of the pressure port and the front of the differential pressure measurement hole The back of each pilot hole and the back of the differential pressure measurement hole in communication with the cannula section is provided with a throttle member, the throttle member blocking the front and rear sides of the pressure port Between the different specifications of the mouth of the mouth have different degrees of throttling the degree of throttling pieces.

2. The disposable differential-pressure-type respiratory flow device as claimed in claim 1, characterized in that the throttle is an orifice plate, a standard nozzle or a perforated plate.

3. The disposable differential-pressure-type respiratory flow device as claimed in claim 1, characterized in that corresponding to the position of the two differential pressure measuring holes, an annular groove is respectively formed in an inner circumferential surface of the fixed pipe in an annular shape, each annular groove is communicated with an inner end of each corresponding differential pressure measuring hole, a plurality of pressure guide holes on the front side are provided and communicated with the pressure differential measuring holes on the front side through the annular groove on the front side and the pressure guide holes on the rear side, there are a plurality of annular grooves communicating with the pressure differential measuring hole on the front side through the rear annular groove.

4. The disposable differential-pressure-type respiratory flow device as claimed in claim 1, characterized in that one or more working holes are arranged on the side around the engaging section in a straight line arrangement of the front and the back interval; the positions of the working holes are matched with each other selectively or not on the outer peripheral surface of the inserting section a combination of two or more of the number and the position of the concavo-convex structure, and each combination only corresponds to the specification of one type of throttling member.

5. The disposable differential-pressure-type respiratory flow device as claimed in claim 4, characterized in that an insertion groove extending in the front-rear direction is recessed formed on an inner peripheral surface of the fixed pipe at a position corresponding to an inner end of each of the working holes, the insertion groove is formed with an opening at a rear end surface of the fixed pipe, and the insertion groove and each working hole communicates with each other; each of the concave-convex structures is a convex structure, and each of the convex structures is slidable along the insertion groove.

6. The disposable differential-pressure-type respiratory flow device as claimed in claim 1, characterized in that a pressure measurement hole is formed through a periphery of the drainage section, and an inner end of the pressure measurement hole communicates with the interior of the drainage section.

7. The disposable differential-pressure-type respiratory flow device as claimed in claim 1, characterized in that a fabric is bonded to the surface of the cannula section, and the fabric covers the pressure guide holes.

8. The disposable differential-pressure-type respiratory flow device as claimed in claim 3, characterized in that corresponding to a plurality of pressure guide holes arranged around the front and rear sides, two shallow grooves are concavely formed on the outer circumferential surface of the cannula section, and a fabric is stuck on each shallow groove to cover the pressure guide hole.

9. The disposable differential-pressure-type respiratory flow device as claimed in claim 1, characterized in that a filter layer is embedded inside the mouthpiece segment.

10. The disposable differential-pressure-type respiratory flow device as claimed in claim 1, characterized in that a straight guiding structure is formed between the inner peripheral surface of the engaging section and the outer peripheral surface of the cannula section, the guiding structure comprises a guiding groove and a guiding protrusion which can slide along the guiding groove.

11. The disposable differential-pressure-type respiratory flow device as claimed in claim 2, characterized in that one or more working holes are arranged on the side around the engaging section in a straight line arrangement of the front and the back interval; the positions of the working holes are matched with each other selectively or not on the outer peripheral surface of the inserting section a combination of two or more of the number and the position of the concavo-convex structure, and each combination only corresponds to the specification of one type of throttling member.

12. The disposable differential-pressure-type respiratory flow device as claimed in claim 11, characterized in that an insertion groove extending in the front-rear direction is recessed formed on an inner peripheral surface of the fixed pipe at a position corresponding to an inner end of each of the working holes, the insertion groove is formed with an opening at a rear end surface of the fixed pipe, and the insertion groove and each working hole communicates with each other; each of the concave-convex structures is a convex structure, and each of the convex structures is slidable along the insertion groove.

13. The disposable differential-pressure-type respiratory flow device as claimed in claim 2, characterized in that a pressure measurement hole is formed through a periphery of the drainage section, and an inner end of the pressure measurement hole communicates with the interior of the drainage section.

14. The disposable differential-pressure-type respiratory flow device as claimed in claim 2, characterized in that a fabric is bonded to the surface of the cannula section, and the fabric covers the pressure guide holes.

15. The disposable differential-pressure-type respiratory flow device as claimed in claim 2, characterized in that a filter layer is embedded inside the mouthpiece segment.

16. The disposable differential-pressure-type respiratory flow device as claimed in claim 2, characterized in that a straight guiding structure is formed between the inner peripheral surface of the engaging section and the outer peripheral surface of the cannula section, the guiding structure comprises a guiding groove and a guiding protrusion which can slide along the guiding groove.

17. The disposable differential-pressure-type respiratory flow device as claimed in claim 3, characterized in that one or more working holes are arranged on the side around the engaging section in a straight line arrangement of the front and the back interval; the positions of the working holes are matched with each other selectively or not on the outer peripheral surface of the inserting section a combination of two or more of the number and the position of the concavo-convex structure, and each combination only corresponds to the specification of one type of throttling member.

18. The disposable differential-pressure-type respiratory flow device as claimed in claim 17, characterized in that an insertion groove extending in the front-rear direction is recessed formed on an inner peripheral surface of the fixed pipe at a position corresponding to an inner end of each of the working holes, the insertion groove is formed with an opening at a rear end surface of the fixed pipe, and the insertion groove and each working hole communicates with each other; each of the concave-convex structures is a convex structure, and each of the convex structures is slidable along the insertion groove.

19. The disposable differential-pressure-type respiratory flow device as claimed in claim 3, characterized in that a pressure measurement hole is formed through a periphery of the drainage section, and an inner end of the pressure measurement hole communicates with the interior of the drainage section.

20. The disposable differential-pressure-type respiratory flow device as claimed in claim 3, characterized in that a fabric is bonded to the surface of the cannula section, and the fabric covers the pressure guide holes.
